(19) United States
(12) Patent Application Publication
(10) Pub. No.: US 2025/0259802 A1
(43) Pub. Date: Aug. 14, 2025

(54) HALOGENATED COMPOUNDS FOR ENERGY STORAGE DEVICE FORMULATIONS

(71) Applicant: Maxwell Technologies Korea Co., Ltd., Yongin-si, Gyeonggi-do (KR)

(72) Inventors: Soon Hyung Kwon, Gyeonggi-do (KR); Jung Ho Choi, Gyeonggi-do (KR); Kyong Hee Kwon, Gyeonggi-do (KR); Sol Seon Oh, Gyeonggi-do (KR); A-Rum Chang, Gyeonggi-do (KR)

(21) Appl. No.: 18/857,644

(22) PCT Filed: Apr. 13, 2023
(86) PCT No.: PCT/US2023/018539

§ 371 Date: Oct. 17, 2024

Related U.S. Application Data
(60) Provisional application No. 63/363,141, filed on Apr. 18, 2022.

(51) Int. Cl. H01G 11/64; C07D 213/61; H01G 11/30; H01G 11/60; H01G 11/62; H01G 11/84

(52) U.S. Cl. CPC H01G 11/64; C07D 213/61; H01G 11/30; H01G 11/60; H01G 11/62; H01G 11/84

(57) ABSTRACT

Performance improvers and formulations for energy storage devices are disclosed. The performance improvers include halogenated aromatic heterocycle compounds that may be utilized to improve the performance of energy storage devices (e.g., capacitors) operating at voltage above 3 V (e.g., 3.2 V).

### INCORPORATION BY REFERENCE TO ANY PRIORITY APPLICATIONS

Any and all applications for which a foreign or domestic priority claim is identified in the Application Data Sheet or PCT Request as filed with the present application are hereby incorporated by reference under 37 CFR 1.57, and Rules 4.18 and 20.6, including U.S. Provisional Application No. 63/363,141, filed Apr. 18, 2022.

### BACKGROUND

### Field

The present invention relates generally to energy storage devices, and specifically to performance improvers, more particularly halogenated compounds, for use in energy storage devices.

### Description of the Related Art

Electrical energy storage cells are widely used to provide power to electronic, electromechanical, electrochemical, and other useful devices. Such cells include primary chemical cells, secondary (rechargeable) cells, fuel cells, and various species of capacitors, including ultracapacitors. Increasing the operating voltage and temperature of energy storage devices, including capacitors, would be desirable for enhancing energy storage, increasing power capability, and broadening real-world use cases.

However, at higher operating temperatures and voltages energy storage devices may undergo undesired processes that result in a reduction in performance, or in outright cell failure. Such processes may include chemical and electrochemical reactions of the electrolyte and/or other components of the device.

Over the life of an energy storage device, deterioration of device performance may manifest as capacitance fade, increased equivalent series resistance (ESR) of the device, self-discharge, pseudocapacitance and/or gas formation. Thus, there is a need for energy storage devices having improved stability under elevated voltage and temperature conditions of operation.

### SUMMARY

For purposes of summarizing the disclosure and the advantages achieved over the prior art, certain objects and advantages of the disclosure are described herein. Not all such objects or advantages may be achieved in any particular embodiment. Thus, for example, those skilled in the art will recognize that the invention may be embodied or carried out in a manner that achieves or optimizes one advantage or group of advantages as taught herein without necessarily achieving other objects or advantages as may be taught or suggested herein.

In one aspect, an electrolyte is described. The electrolyte comprises: a salt; a solvent; and a performance improver, wherein the performance improver comprises a halogenated heteroaryl compound.

In some embodiments, the halogenated heteroaryl compound comprises a compound of Formula (I) having the structure:

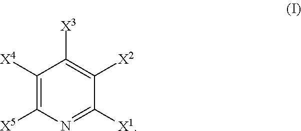

wherein each X¹, X², X³, X⁴ and X⁵ is independently H or a halogen, provided that at least one of X¹, X², X³, X⁴ and X⁵ is a halogen. In some embodiments, the halogen is selected from the group consisting of —F, —Cl, —Br, and —I.

In some embodiments, the compound of Formula (I) is a compound selected from the group consisting of:

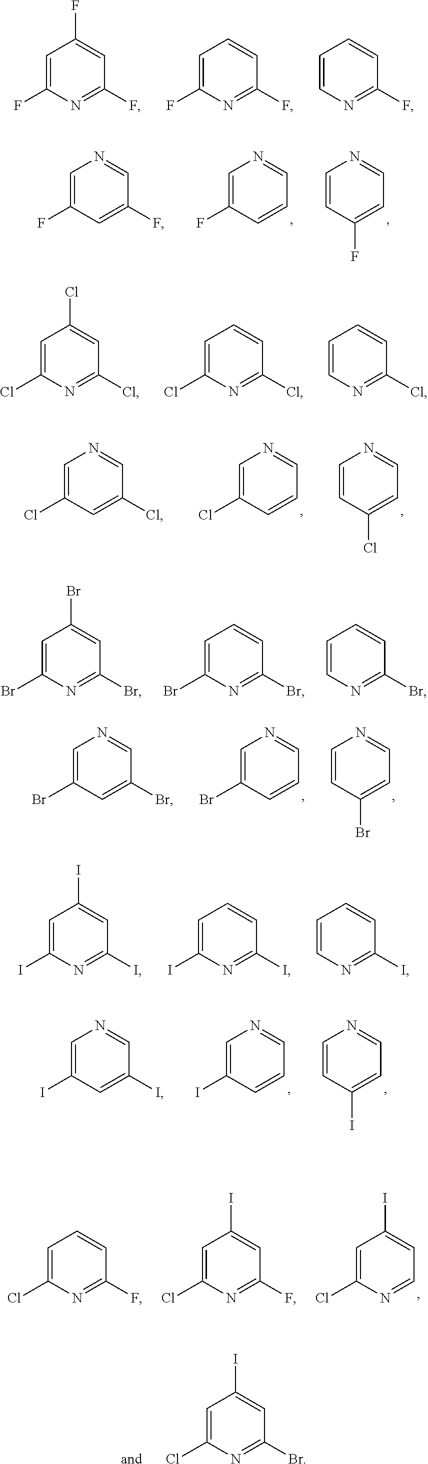

The electrolyte of any one of Claims 2-4, wherein the compound of Formula (I) is selected from the group consisting of:

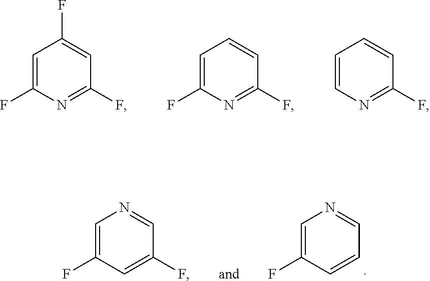

In some embodiments, the compound of Formula (I) is

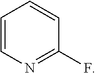

In some embodiments, the compound of Formula (I) is

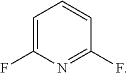

In some embodiments, the compound of Formula (I) is

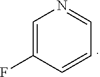

In some embodiments, the electrolyte comprises about 0.1 wt % to about 30 wt % of the performance improver. In some embodiments, the electrolyte comprises about 0.5 wt % to about 15 wt % of the compound of Formula (1). In some embodiments, the salt comprises a compound selected from the group consisting of a tetraethyl ammonium salt, a triethylmethyl ammonium salt,, a methyltriethylammonium salt, a tetrabutylammonium salt, a tetraethylphosphonium salt, a tetrapropylphosphonium salt, a tetrabutylphosphonium salt, a tetrahexylphosphonium salt, a lithium salt, a trimethylethyl ammonium (TMEA) salt, a diethyldimethyl ammonium (DEDMA) salt, a diethyl-methylmethoxyethyl ammonium (DEME) salt, a Tetramethyl ammonium (TMA) salt, a piperidine-1-spiro-1′-pyrrolidinium (SPP) salt, a spiro-(1,1′)-bipyrrolidinium (SBP) salt, a 1,1-Dimethylpyrrolidinium (DMP) salt, a Dimethylammonium (DMA) salt, a 1-Ethyl-1-methylpyrrolidinium (EMP) salt, a 1-Methyl-1-propylpyrrolidinium (MPP) salt, a 1-Butyl-1-methylpyrrolidinium (BMP) salt, a 1-Ethyl-3- methylimidazolium (EMI) salt, a 1-Methyl-3-propylimidazolium (PMI) salt, a 1-Butyl-3-methylimidazolium (BMI) salt, and combinations thereof. In some embodiments, the solvent comprises a compound selected from the group consisting of acetonitrile, gamma-butyrolactone, dimethoxyethane, N,N,-dimethylformamide, hexamethyl-phosphorotriamide, tetrahydrofuran, 2-methyltetra-hydrofuran, dimethyl sulfoxide, dimethyl sulfite, sulfolane, nitromethane, dioxolane, ethylene carbonate (EC), propylene carbonate (PC), vinyl ethylene carbonate (VEC), vinylene carbonate (VC), fluoroethylene carbonate (FEC), dimethyl carbonate (DMC), diethyl carbonate (DEC), ethyl methyl carbonate (EMC), capronitrile, valeronitrile, butyronitrile, propionitrile, methyl ethyl sulfone, methyl isopropyl sulfone, ethyl isobutyl sulfone, isopropyl-s-butyl sulfone, butyl isobutyl sulfone, and combinations thereof.

In another aspect, an energy storage device is described. The energy storage device comprises: a cathode; an anode; a separator disposed between the cathode and the anode; an electrolyte comprising a salt, a solvent and a performance improver; and a housing, wherein the cathode, anode, separator and electrolyte are disposed within the housing.

In another aspect, an energy storage device is described. The energy storage device comprises: a cathode; an anode; a separator disposed between the cathode and the anode; the electrolyte; and a housing, wherein the cathode, anode, separator and electrolyte are disposed within the housing.

In some embodiments, the energy storage device is a capacitor. In some embodiments, the energy storage device is configured to maintain at least about 80% of its initial capacitance when operating at about 3.2 V for a period of about 1000 hours at a temperature of about 65° C. In some embodiments, the energy storage device is configured to grow at most about 4% of its initial length when operating at about 3.2 V for a period of about 1000 hours at a temperature of about 65° C. In some embodiments, the energy storage device is configured to maintain at most about 175% of its initial DC-ESR when operating at about 3.2 V for a period of about 1000 hours at a temperature of about 65° C. In some embodiments, the energy storage device is configured to maintain at most about 175% of its initial AC-ESR when operating at about 3.2 V for a period of about 1000 hours at a temperature of about 65° C.

In another aspect, a method of fabricating an energy storage device is disclosed. The method comprises: disposing a cathode, an anode, a separator, an electrolyte and a performance improver within a housing, wherein the performance improver comprises a compound of Formula (II) having the structure:

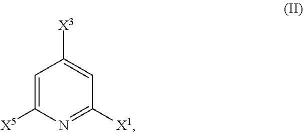

wherein each X¹, X³, and X⁵ is independently H or a halogen, provided that at least one of X¹, X³ and X⁵ is a halogen.

In another aspect, a method of fabricating an energy storage device is disclosed. The method comprises: disposing a cathode, an anode, a separator, an electrolyte and a performance improver within a housing, wherein the performance improver comprises a halogenated heteroaryl compound.

In some embodiments, prior to the disposing step at least one of the cathode and anode comprises the performance improver. In some embodiments, prior to the disposing step the electrolyte comprises the performance improver.

In another aspect, an electrode film is described. The electrode film comprises: an active material; a binder; and a performance improver, wherein the performance improver comprises a halogenated heteroaryl compound.

All of these embodiments are intended to be within the scope of the invention disclosed herein. These and other embodiments will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1 shows a perspective view illustration of an energy storage device according to an embodiment.

FIG. 2A shows an exploded view illustration of the energy storage device of FIG. 1.

FIG. 2B shows an exploded view illustration of the electrode unit of FIG. 2A.

FIG. 3A shows a line graph detailing the change in capacitance over time between an energy storage device with a performance improver according to some embodiments, compared to control and comparative energy storage devices.

FIG. 3B shows a line graph detailing the change in cell length over time between an energy storage device with a performance improver according to some embodiments, compared to control and comparative energy storage devices.

FIG. 3C shows a line graph detailing the change in the direct current equivalent series resistance (DC-ESR) over time between an energy storage device with a performance improver according to some embodiments, compared to control and comparative energy storage devices.

FIG. 4A shows a line graph detailing the change in cell length over time between an energy storage device with a performance improver according to some embodiments, compared to a control energy storage device.

FIG. 4B shows a line graph detailing the change in capacitance over time between an energy storage device with a performance improver according to some embodiments, compared to a control energy storage device.

FIG. 4C shows a line graph detailing the change in the direct current equivalent series resistance (DC-ESR) over time between an energy storage device with a performance improver according to some embodiments, compared to a control energy storage device.

FIG. 4D shows a line graph detailing the change in the alternating current equivalent series resistance (AC-ESR) over time between an energy storage device with a performance improver according to some embodiments, compared to a control energy storage device.

FIG. 5A shows a line graph detailing the change in cell length over time between energy storage devices with performance improvers according to some embodiments, compared to a control energy storage device.

FIG. 5B shows a line graph detailing the change in capacitance over time between energy storage devices with performance improvers according to some embodiments, compared to a control energy storage device.

FIG. 5C shows a line graph detailing the change in the direct current equivalent series resistance (DC-ESR) over time between energy storage devices with performance improvers according to some embodiments, compared to a control energy storage device.

FIG. 6A shows a line graph detailing the change in cell length over time between energy storage devices with various amounts performance improvers according to some embodiments, compared to a control energy storage device.

FIG. 6B shows a line graph detailing the change in capacitance over time between energy storage devices with various amounts performance improvers according to some embodiments, compared to a control energy storage device.

FIG. 6C shows a line graph detailing the change in the alternating current equivalent series resistance (AC-ESR) over time between energy storage devices with various amounts performance improvers according to some embodiments, compared to a control energy storage device.

FIG. 6D shows a line graph detailing the change in the direct current equivalent series resistance (DC-ESR) over time between energy storage devices with various amounts performance improvers according to some embodiments, compared to a control energy storage device.

These and other features, aspects, and advantages of the present disclosure are described with reference to the drawings of certain embodiments, which are intended to illustrate certain embodiments and not to limit the invention.

### DETAILED DESCRIPTION

Although certain embodiments and examples are described herein, those of skill in the art will appreciate that the invention extends beyond the specifically disclosed embodiments and/or uses and obvious modifications and equivalents thereof. Thus, it is intended that the scope of the invention herein disclosed should not be limited by any particular embodiments described herein.

Halogenated heteroaryl performance improvers for use in energy storage devices (e.g., capacitors and/or batteries) are disclosed. In some embodiments, an energy storage device comprises one or more halogenated heteroaryl performance improver compounds as provided herein. In some embodiments, energy storage devices that include such performance improvers demonstrate improved performances, for example such as decreased gas generation, improved capacitance reduction rates and an improved resistance increase rate. In some embodiments, improved energy storage device performances may be realized at operating voltages of at least or greater than 3 V (e.g., about 3.2 V) and/or at operating temperatures of at most or less than 65° C. (e.g., at most about 65° C.) or at operating temperatures of at least or greater than 65° C. (e.g., at least about 65° C.).

In some embodiments, the halogenated heteroaryl performance improver compound may comprise a nitrogen-containing aromatic heterocycle with at least one halogen atom bonded to the aromatic ring. In some embodiments, the nitrogen-containing aromatic heterocycle can be, for example, a pyridine, a pyrimidine, a pyrazine, or a pyridazine. Additional details on halogenated heteroaryl compounds can be found herein.

### Definitions

As used herein, the terms “battery” and “capacitor” are to be given their ordinary and customary meanings to a person of ordinary skill in the art. The terms “battery” and “capacitor” are nonexclusive of each other. A capacitor or battery can refer to a single electrochemical cell that may be operated alone, or operated as a component of a multi-cell system.

As used herein, capacitance (F—Farads) is a measurement of energy storage in joules. C=Q/V

As used herein, the voltage is the maximum operating voltage for a single capacitor. The rated voltage is the voltage at which the performance data is measured. It is possible for the capacitors to experience voltages in excess of the rated voltage. The impact is dependent on the time and temperature during this exposure.

As used herein, surge voltage is the maximum voltage that an ultracapacitor can operate at for a short periods of time with minimal damage or cell opening.

As used herein, internal resistance can be measured through directing current equivalent series resistance (DC-ESR) and/or alternating current equivalent series resistance (AC-ESR). DC-ESR is measured at a constant current charge of 10 mA/F to a V_{R}, and a 5 minute hold at V_{R}. AC-ESR is measured at an amplitude of 5 to 10 mV and a frequency of 50 to 1 kHz.

### Energy Storage Devices

As provided herein, an energy storage device can be a capacitor, a hybrid capacitor (e.g., a lithium-ion capacitor (LIC)), a supercapacitor, an ultracapacitor, and/or a battery. The energy storage device can be characterized by an operating voltage. Performance improvers described herein (e.g., halogenated heteroaryl performance improvers), as well as an electrolyte and/or electrode including a performance improver described herein, can be used in various embodiments with any of a number of energy storage devices, such as one or more batteries, capacitors, capacitor-battery hybrids, fuel cells, or other energy storage systems or devices and combinations thereof. In some embodiments, a performance improver, electrolyte including a performance improver, and/or electrode/electrode film including a performance improver described herein may be implemented in lithium-ion capacitors, lithium-ion batteries, or ultracapacitors.

An energy storage device includes one or more electrodes. An electrode generally includes an electrode film and a current collector. The electrode film can be formed from a mixture of one or more binders and active electrode material. In some embodiments, the electrode and/or electrode film further comprises a performance improver (e.g., a halogenated heteroaryl performance improvers). The one or more binders can be selected from a fluorinated binder (e.g., polytetrafluoroethylene (PTFE), polyvinylidene fluoride (PVDF), a PVDF co-polymer), ultra-high molecular weight polyethylene (UHMWPE), poly(ethylene oxide) (PEO), carboxymethylcellulose (CMC), poly(acrylic Acid) (PAA), a poly(acrylic acid) (PAAX; where X=Li, Na, K, NH₄), poly(vinyl alcohol) (PVA), polyvinyl acetate (PVAc), poly(vinyl alcohol)-poly(acrylic acid) (PAA-PVA), poly(vinyl alcohol)-poly(ethylene imine) (PVA-PEI), poly(acrylic acid)-sodium carboxymethylcellulose (PAA-CMC), poly(acrylic acid sodium)-grafted-carboxymethyl cellulose (NaPAA-g-CMC), poly(acrylic acid)-dially ether (PAA-dially ether), carboxymethyl chitosan (C-chitosan), chitosan-glutaraldehyde (CS-GA), an alginate salt (e.g., sodium alginate, calcium alginate), alginate-carboxymethyl chitosan, catechol conjugated poly(acrylic Acid) (PAA-C), a β-cyclodextrin polymer, carbonyl-β-cyclodextrin, a β-Cyclodextrin polymer with quaternary ammonium cations (β-CDp-N⁺), gum arabic (GA), gum arabic-polyacrylic acid (GA-PAA), gellan gum, xanthan gum (XG), guar gum (GG), xanthan gum-guar gum (XG-GG), locust bean gum (LBG), poly(3,4-ethylenedioxythiophene): poly(styrene4-sulfonate) (PEDOT:PSS), karaya gum (KG), a polyacrylic latex (e.g., LA132), poly(acrylamide)-co-diallyldimethylammonium chloride (AMAC), polyamido amine (PAMAM), gelatin, styrene butyl rubber (SBR), a polyvinylpyrrolidone, a polyacrylonitrile, a polyvinyl alcohol polyolefin, a polyimide, a polyether ketone, a polyepoxide, a polyurethane, or other suitable fibrillizable materials, or combinations thereof.

In some embodiments, the active electrode material is selected from activated carbon, a lithiated carbon, a lithium metal oxide and a metal oxide. In some embodiments, the active electrode material may comprise a carbon based material. The carbon based materials may be selected from activated carbon, a graphene-based carbon (e.g., graphene), a graphite-based carbon (e.g., graphite), a carbon nanotube, and combinations thereof. The lithiated carbon based materials may be selected from lithiated activated carbon, a lithiated graphene-based carbon (e.g., lithiated graphene), a lithiated graphite-based carbon (e.g., lithiated graphite), a lithiated carbon nanotube, and combinations thereof. In some embodiments, the metal oxide has the formula MₓO_{y}. In some embodiments, the metal “M” of the metal oxide is selected from Fe, Mn, Ni, Mo, Cu, Co, Al, Ti, V, and Cr, or a combination thereof. In some embodiment, the lithium metal oxide is selected from Lithium cobalt oxide (LCO), Lithium nickel oxide (LNO), Lithium manganese oxide (LMO), Lithium nickel cobalt manganese oxide (NCM or NMC), Lithium nickel cobalt aluminium oxide (NCA), Lithium iron phosphate (LFP), Lithium iron manganese phosphate (LFMP), Lithium cobalt phosphate (LCP), Lithium iron fluorosulfate (LFSF), Lithium vanadium fluorosulfate (LVSF), Lithium titanium oxide (LTO), Lithium titanium sulfide (LTS), Lithium nickel cobalt manganese oxide (HE-NCM), Lithium maganese oxide (HV-spinel) and Lithium silicon oxide (LSO). In some embodiments, the electrode film further comprises a conductive additive. In some embodiments, the conductive additive is selected from a conductive carbon (e.g., carbon black).

An energy storage device as provided herein can be of any suitable configuration, for example planar, spirally wound, button shaped, or pouch. An energy storage device as provided herein can be a component of a system, for example, a power generation system, an uninterruptible power source systems (UPS), a photo voltaic power generation system, an energy recovery system for use in, for example, industrial machinery and/or transportation. An energy storage device as provided herein may be used to power various electronic device and/or motor vehicles, including hybrid electric vehicles (HEV), plug-in hybrid electric vehicles (PHEV), and/or electric vehicles (EV).

Limiting gas generation during operation of an energy storage device is an important operating parameter. Gas production is undesirable since a sealed cell will become pressurized, reducing cell efficiency and creating a potential hazard. Such devices generally have a housing, such as a metal container or can, which can withstand substantial internal pressure build-up due to gas formation. However, operation at high voltages or temperatures can generate so much gas that an overpressure condition is created. An overpressure condition may be mitigated for example, by opening a safety vent, which releases the built-up pressure. However, opening such a vent may render the device inoperable. Gas formation can also prevent a pouch or other non-rigid container configuration from being used, or can reduce the increased density of active material that would otherwise be provided by a pouch configuration, relative to a spiral bound or other device with a rigid container. Thus, mitigation of gas formation may be desirable. In some embodiments, a performance improver provided herein can reduce gas generation in an energy storage device.

An energy storage device described herein may advantageously be characterized by reduced equivalent series resistance over the life of the device, which may provide a device with increased power density. In some embodiments, capacitors described herein may be characterized by reduced loss of capacitance over the life of the device. Further improvements that may be realized in various embodiments include improved device performances, including improved capacitance stability, and reduced capacitance fade.

Such advantageous improvements performance, such as decreased gas generation, improved capacitance reduction rates and an improved resistance increase rate, may be achieved in energy storage devices utilizing the performance improvers disclosed even when operating at increased voltages, increased temperatures, extended lifetimes and/or extended cycles. In some embodiments, secondary electrochemical reactions of the performance improvers, electrolyte and/or electrodes are reduced in energy storage devices utilizing the performance improvers described herein. In some embodiments, an energy storage device is configured to have an operating voltage of, of about, of at least, or of at least about, 2 V, 2.1 V, 2.2 V, 2.5 V, 2.6 V, 2.7 V, 2.8 V, 2.9 V, 3 V, 3.1 V, 3.2 V, 3.3 V, 3.4 V, 3.5 V, 3.6 V, 3.7 V, 3.8 V or 4 V, or any range of values therebetween. For example, in some embodiments the energy storage device may have an operating voltage of about 2.2 V to about 3.8 V, of about 3.2 V to about 3.3 V, or of about 3.0 V to about 3.3 V. In additional examples, in some embodiments the energy storage device may have an operating voltage of at least 3.2 V, of at least 3.0 V, or of at least 2.7 V. In some embodiments, an energy storage device is configured to have an operating temperature of, of about, of at most, or of at most about, 0° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C. or 100° C., or any range of values therebetween. In some embodiments, an energy storage device is configured for operation at any selected operating voltages and/or temperatures described herein. For example, in some embodiments the energy storage device is configured for continual operation at 2.7 V at 25-85° C., 2.8 V at 25-85° C., 2.9 V at 25-85° C., 3 V at 25-85° C., 3.1 V at 25-85° C., 3.2 V at 25-85° C., 3.3 V at 25-85° C., 3.4 V at 25-85° C., 3.5 V at 25-85° C., 3.6 V at 25-85° C., 3.7 V at 25-85° C., 3.8 V at 25-85° C., 3.9 V at 25-85° C., 4.0 V at 25-85° C., or 4.1 V at 25-85° C. In some embodiments, the energy storage device is configured for operation at about 3.2 V and about 85 C, about 3.2 V and about 80° C., about 3.3 V and about 75° C., about 3.2 V and about 75° C., about 3.1 V and about 75° C., about 3 V and about 75° C., about 3.2 V and about 65° C., about 3.3 V and about 65° C. or about 3.2 V and about 25° C.

An energy storage device may include one or more technologies described herein (e.g., a balogenated heteroaryl performance improver) to enable improved performances. For example, in some embodiments the energy storage device is configured to maintain a capacitance at least about 70% of its initial capacitance, at most 6% of its initial length, at most 400% of its initial direct current equivalent series resistance (DC-ESR) and/or at most 250% of its initial alternating current equivalent series resistance (AC-ESR) when operating at a voltage of 3.2 V over a period of about 1000 hours and at a temperature of, of about, of at least, or at least about, 65° C. In some embodiments, the energy storage device is configured to maintain a capacitance at least about 80% of its initial capacitance, at most 4% of its initial length, at most 200% of its initial DC-ESR and/or at most 200% of its initial AC-ESR when operating at a voltage of 3.2 V over a period of about 150 hours and at a temperature of, of about, of at least, or at least about, 75° C.

In some embodiments, the energy storage device is configured to maintain at least, or at least about, 65%, 70%, 75%, 80%, 85%, 90%, 92%, 95%, 98% or 99% of its. initial capacitance when operating at, or at about, 3.2 V for a period of, of about, of at least, or at least about, 1000 hours at, at about, at least, or at least about, 65° C. In some embodiments, the energy storage device is configured to maintain at least, or at least about, 65%, 70%, 75%, 80%, 85%, 90%, 92%, 95%, 98% or 99% of its initial capacitance when operating at, or at about, 3.2 V for a period of, of about, of at least, or at least about, 2000 hours at, at about, at least, or at least about, 65° C. In some embodiments, the energy storage device is configured to maintain at least, or at least about, 65%, 70%, 75%, 80%, 85%, 90%, 92%, 95%, 98% or 99% of its initial capacitance when operating at, or at about, 3.2 V for a period of, of about, of at least, or at least about, 10 years at, at about, at least, or at least about, 25° C. In some embodiments, the energy storage device is configured to maintain at least, or at least about, 75%, 80%, 85%, 90%, 92%, 95%, 98% or 99% of its initial capacitance when operating at, or at about, 3.2 V for a period of, of about, of at least, or at least about, 150 hours at, at about, at least, or at least about, 75° C.

In some embodiments, the energy storage device is configured to grow at most, or at most about, 10%, 8%, 7%, 6%, 5%, 4%, 3%, 2%, 1%, 0.5% or 0.1% of its initial length when operating at, or at about, 3.2 V for a period of, of about, of at least, or at least about, 1000 hours at, at about, at least, or at least about, 65° C. In some embodiments, the energy storage device is configured to grow at most, or at most about, 10%, 8%, 7%, 6%, 5%, 4%, 3%, 2%, 1%, 0.5% or 0.1% of its initial length when operating at, or at about, 3.2 V for a period of, of about, of at least, or at least about, 2000 hours at, at about, at least, or at least about, 65° C. In some embodiments, the energy storage device is configured to grow at most, or at most about, 10%, 8%, 7%, 6%, 5%, 4%, 3%, 2%, 1%, 0.5% or 0.1% of its initial length when operating at, or at about, 3.2 V for a period of, of about, of at least, or at least about, 10 years at, at about, at least, or at least about, 25° C. In some embodiments, the energy storage device is configured to grow at most, or at most about, 8%, 7%, 6%, 5%, 4%, 3%, 2%, 1%, 0.5% or 0.1% of its initial length when operating at, or at about, 3.2 V for a period of, of about, of at least, or at least about, 150 hours at, at about, at least, or at least about, 75° C.

In some embodiments, the energy storage device is configured to maintain at most, or at most about, 500%, 450%, 400%, 350%, 300%, 250%, 200%, 175%, 150% or 100% of its initial DC-ESR when operating at, or at about, 3.2 V for a period of, of about, of at least, or at least about, 1000 hours at, at about, at least, or at least about, 65° C. In some embodiments, the energy storage device is configured to maintain at most, or at most about, 500%, 450%, 400%, 350%, 300%, 250%, 200%, 175%, 150% or 100% of its initial DC-ESR when operating at, or at about, 3.2 V for a period of, of about, of at least, or at least about, 2000 hours at, at about, at least, or at least about, 65° C. In some embodiments, the energy storage device is configured to maintain at most, or at most about, 500%, 450%, 400%, 350%, 300%, 250%, 200%, 175%, 150% or 100% of its initial DC-ESR when operating at, or at about, 3.2 V for a period of, of about, of at least, or at least about, 10 years at, at about, at least, or at least about, 25° C. In some embodiments, the energy storage device is configured to maintain at most, or at most about, 300%, 250%, 200%, 175%, 150%, 125%, 100%, 75%, 50% or 25% of its initial DC-ESR when operating at, or at about, 3.2 V for a period of, of about, of at least, or at least about, 150 hours at, at about, at least, or at least about, 75° C.

In some embodiments, the energy storage device is configured to maintain at most, or at most about, 300%, 250%, 200%, 175%, 150%, 125%, 100%, 75%, 50% or 25% of its initial AC-ESR when operating at, or at about, 3.2 V for a period of, of about, of at least, or at least about, 1000 hours at, at about, at least, or at least about, 65° C. In some embodiments, the energy storage device is configured to maintain at most, or at most about, 300%, 250%, 200%, 175%, 150%, 125%, 100%, 75%, 50% or 25% of its initial AC-ESR when operating at, or at about, 3.2 V for a period of, of about, of at least, or at least about, 2000 hours at, at about, at least, or at least about, 65° C. In some embodiments, the energy storage device is configured to maintain at most, or at most about, 300%, 250%, 200%, 175%, 150%, 125%, 100%, 75%, 50% or 25% of its initial AC-ESR when operating at, or at about, 3.2 V for a period of, of about, of at least, or at least about, 10 years at, at about, at least, or at least about, 25° C. In some embodiments, the energy storage device is configured to maintain at most, or at most about, 300%, 250%, 200%, 175%, 150%, 125%, 100%, 75%, 50% or 25% of its initial AC-ESR when operating at, or at about, 3.2 V for a period of, of about, of at least, or at least about, 150 hours at, at about, at least, or at least about, 75° C.

In some embodiments, energy storage devices may be operated at a constant current charge and/or discharge from an operating voltage to half of the operating voltage at an operating temperature. For example, an energy storage device (e.g., capacitor) may be operated from 3.2 V to 1.6 V at a temperature of 25° C., 65° C. or 75° C.

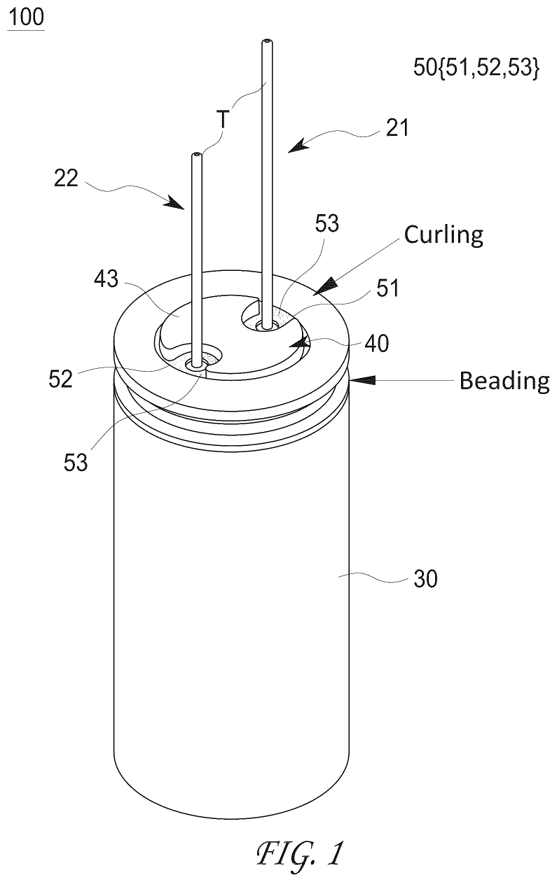

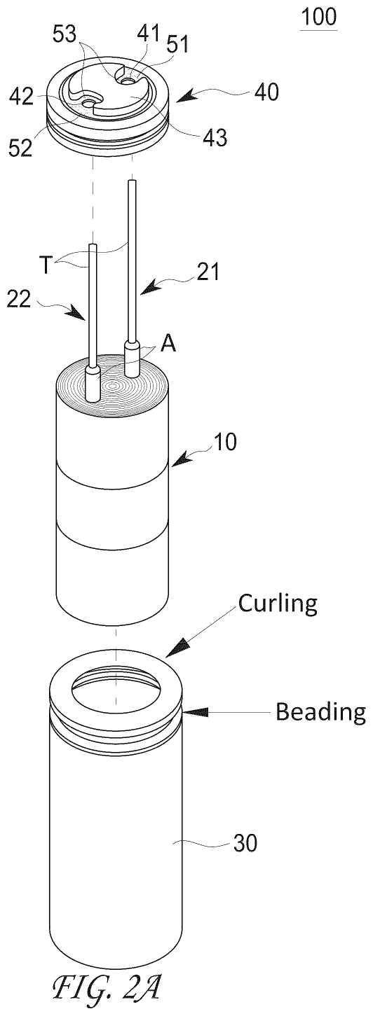

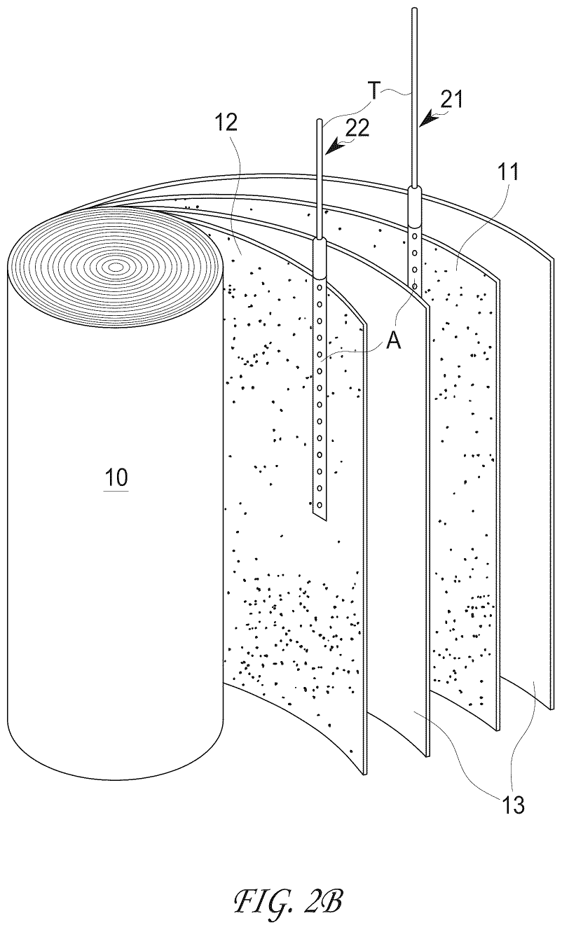

FIG. 1 shows an energy storage device 100 according to an embodiment. FIG. 2A is an exploded view of the energy storage device 100 of FIG. 1. FIG. 2B is an exploded view of the electrode unit 10 of FIG. 2A. The energy storage device 100 can be an electric double layer device such as an electric double layer capacitor (EDLC) or an ultra-capacitor. The energy storage device 100 includes an electrode unit 10, a first terminal 21, a second terminal 22, a case 30 and a closure 40. The electrode unit 10 includes a first current collection sheet 11, a second current collection sheet 12 and separating sheets 13. The electrode unit 10 can be of a winding type where the first and second current collection sheets 11 and 12 are wound while being separated from each other by the separating sheet 13. The first and second terminals 21 and 22 are respectively connected to the first and second current collection sheets 11 and 12.

The first current collection sheet 11 and the second current collection sheet 12 may be used respectively as a positive electrode current collector and a negative electrode current collector. In this embodiment, the first terminal 21 is connected to the first current collection sheet 11 (used as a positive electrode current collector) and functions as a positive electrode terminal. Furthermore, the second terminal 22 is connected to the second current collection sheet 12 (used as a negative electrode current collector) and functions as a negative electrode terminal. Each of the first and second current collection sheets 11 and 12 may be made of aluminum foil on which an electrode active material is coated. The electrode active material may be conductive paste including mostly activated carbon. In some embodiments, as shown in FIG. 2A and 2B, each of the first and second terminals 21 and 22 includes an aluminum terminal A.

The case 30 receives and accommodates the electrode unit 10. The closure 40 covers a top portion of the case 30. The electrode unit 10 is impregnated with an electrolyte and is placed in the case 30.

### Electrolytes

As described herein, energy storage devices include an electrolyte that is capable of transporting ions between a positive electrode and a negative electrode. The electrolyte may be a solution having a solvent, a salt and a performance improver (e.g., an additive), with the salt providing ionic species for ionic conductivity and contact between the positive electrode and the negative electrode. A suitable electrolyte may also exhibit a low viscosity and/or a high degree of ionic conductivity, thereby enabling a decreased capacitor internal resistance and increased capacitor voltage during charging and discharging of the capacitor. For example, an increased solubility of the salt in the solvent may enable increased ionic conductivity between the positive the negative electrodes. A suitable electrolyte may exhibit chemical and/or electrochemical stability under the operating conditions of the energy storage device and may be able to withstand repeated charge discharge cycles of the ultracapacitor.

In some embodiments, the solvent can include a liquid solvent. A solvent as provided herein need not dissolve every component, and need not completely dissolve any component, of the electrolyte. In further embodiments, the solvent can be an organic solvent. In some embodiments, a solvent can include one or more functional groups selected from carbonates, ethers and/or esters. In some embodiments, the solvent can comprise a carbonate (e.g., a cyclic carbonate, an acyclic carbonate). In some embodiments, the solvent is selected from acetonitrile, gamma-butyrolactone, dimethoxyethane, N,N,-dimethylformamide, hexamethyl-phosphorotriamide, tetrahydrofuran, 2-methyltetra-hydrofuran, dimethyl sulfoxide, dimethyl sulfite, sulfolane, nitromethane, dioxolane, ethylene carbonate (EC), propylene carbonate (PC), vinyl ethylene carbonate (VEC), vinylene carbonate (VC), fluoroethylene carbonate (FEC), dimethyl carbonate (DMC), diethyl carbonate (DEC), etbyl methyl carbonate (EMC), capronitrile, valeronitrile, butyronitrile, propionitrile, methyl ethyl sulfone, methyl isopropyl sulfone, ethyl isobutyl sulfone, isopropyl-s-butyl sulfone, butyl isobutyl sulfone, and combinations thereof.

In some embodiments, the salt is selected from a tetraethyl ammonium salt (e.g., tetraethyl ammonium tetrafluoroborate, tetraethyl ammonium iodide, tetraethyl ammonium hexafluorophosphate, tetraethyl ammonium bis(trifluoromethanesulfonyl)imide (TFSI), tetraethyl ammonium bis(fluorosulfonyl)imide (FSI)), a triethylmethyl ammonium salt (e.g., triethylmethyl ammonium tetrafluoroborate, triethylmethyl ammonium iodide, triethylmethyl ammonium hexafluorophosphate, triethylmethyl ammonium bis(trifluoromethanesulfonyl)imide (TFSI), triethylmethyl ammonium bis(fluorosulfonyl)imide (FSI)), a methyltriethylammonium salt (e.g., methyltriethylammonium tetrafluoroborate), a tetrabutylammonium salt (e.g., tetrabutylammonium tetrafluoroborate), a tetraethylphosphonium salt (e.g., tetraethylphosphonium tetrafluoroborate, tetraethylphosphonium hexafluorophosphate, tetraethylphosphonium trifluoromethylsulfonate,), a tetrapropylphosphonium salt (e.g., tetrabutylphosphonium tetrafluoroborate), a tetrapropylphosphonium salt (e.g., tetrabutylphosphonium tetrafluoroborate), a tetrahexylphosphonium salt (e.g., tetrahexylphosphonium tetrafluoroborate), a lithium salt (e.g., lithium tetrafluoroborate, lithium hexafluorophosphate, lithium trifluoromethylsulfonate, lithium perchlorate, lithium bis(trifluoromethansulfonyl)imide, lithium trifluoromethansulfonate), a trimethylethyl ammonium (TMEA) salt (e.g., trimethylethyl ammonium tetrafluoroborate, trimethylethyl ammonium hexafluorophosphate, trimethylethyl ammonium bis(trifluoromethanesulfonyl)imide, trimethylethyl ammonium bis(fluorosulfonyl)imide), a diethyldimethyl ammonium (DEDMA) salt (e.g., diethyldimethyl ammonium tetrafluoroborate, diethyldimethyl ammonium hexafluorophosphate, diethyldimethyl ammonium bis(trifluoromethanesulfonyl)imide, diethyldimethyl ammonium bis(fluorosulfonyl)imide), a diethyl-methylmethoxyethyl ammonium (DEME) salt (e.g., diethyl-methylmethoxyethyl ammonium tetrafluoroborate, diethyl-methylmethoxyethyl ammonium hexafluorophosphate, diethyl-methylmethoxyethyl ammonium bis(trifluoromethanesulfonyl)imide, dicthyl-methylmethoxyethyl ammonium bis(fluorosulfonyl)imide), a Tetramethyl ammonium (TMA) salt (e.g., Tetramethyl ammonium tetrafluoroborate, Tetramethyl ammonium hexafluorophosphate, Tetramethyl ammonium bis(trifluoromethanesulfonyl)imide, Tetramethyl ammonium bis(fluorosulfonyl)imide), a piperidine-1-spiro-1′-pyrrolidinium (SPP) salt (e.g., piperidine-1-spiro-1′-pyrrolidinium tetrafluoroborate, piperidine-1-spiro-1′-pyrrolidinium hexafluorophosphate, piperidine-1-spiro-1′-pyrrolidinium bis(trifluoromethanesulfonyl)imide, piperidine-1-spiro-1′-pyrrolidinium bis(fluorosulfonyl)imide), a spiro-(1,1′)-bipyrrolidinium (SBP) salt (e.g., spiro-(1,1′)-bipyrrolidinium tetrafluoroborate, spiro-(1,1′)-bipyrrolidinium iodide, spiro-(1,1 ‘)-bipyrrolidinium hexafluorophosphate, spiro-(1,1’)-bipyrrolidinium bis(trifluoromethylsulfonyl)imide, spiro-(1,1′)-bipyrrolidinium bis(fluorosulfonyl)imide)), a 1,1-Dimethylpyrrolidinium (DMP) salt (e.g., 1,1-Dimethylpyrrolidinium tetrafluoroborate, 1,1-Dimethylpyrrolidinium hexafluorophosphate, 1,1-Dimethylpyrrolidinium bis(trifluoromethylsulfonyl)imide, 1,1-Dimethylpyrrolidinium bis(fluorosulfonyl)imide), a Dimethylammonium (DMA) salt (e.g., dimethylammonium tetrafluoroborate, Dimethylammonium hexafluorophosphate, Dimethylammonium bis(trifluoromethylsulfonyl)imide, Dimethylammonium bis(fluorosulfonyl)imide), a 1-Ethyl-1-methylpyrrolidinium (EMP) salt (e.g., 1-Ethyl-1-methylpyrrolidinium tetrafluoroborate, 1-Ethyl-1-methylpyrrolidinium hexafluorophosphate, 1-Ethyl-1-methylpyrrolidinium bis(trifluoromethanesulfonyl)imide, 1-Ethyl-1-methylpyrrolidinium bis(fluorosulfonyl)imide), a 1-Methyl-1-propylpyrrolidinium (MPP) salt (e.g., 1-Methyl-1-propylpyrrolidinium tetrafluoroborate, 1-Methyl-1-propylpyrrolidinium hexafluorophosphate, 1-Methyl-1-propylpyrrolidinium bis(trifluoromethanesulfonyl)imide, 1-Methyl-1-propylpyrrolidinium bis(fluorosulfonyl)imide), a 1-Butyl-1-methylpyrrolidinium (BMP) salt (e.g., 1-Butyl-1-methylpyrrolidinium tetrafluoroborate, 1-Butyl-1-methylpyrrolidinium hexafluorophosphate, 1-Butyl-1-methylpyrrolidinium bis(trifluoromethanesulfonyl)imide, 1-Butyl-1-methylpyrrolidinium bis(fluorosulfonyl)imide), a 1-Ethyl-3-methylimidazolium (EMI) salt (e.g., 1-Ethyl-3-methylimidazolium tetrafluoroborate, 1-Ethyl-3-methylimidazolium hexafluorophosphate, 1-Ethyl-3-methylimidazolium bis(trifluoromethanesulfonyl)imide, 1-Ethyl-3-methylimidazolium bis(fluorosulfonyl)imide), a 1-Methyl-3-propylimidazolium (PMI) salt (e.g., 1-Methyl-3-propylimidazolium tetrafluoroborate, 1-Methyl˜3-propylimidazolium hexafluorophosphate, 1-Methyl-3-propylimidazolium bis(trifluoromethanesulfonyl)imide, 1-Methyl-3-propylimidazolium bis(fluorosulfonyl)imide), a 1-Butyl-3-methylimidazolium (BMI) salt (e.g., 1-Butyl-3-methylimidazolium tetrafluoroborate, 1-Butyl-3-methylimidazolium hexafluorophosphate, 1-Butyl-3-methylimidazolium bis(trifluoromethanesulfonyl)imide, 1-Butyl-3-methylimidazolium bis(fluorosulfonyl)imide), and combinations thereof. In some embodiments, the electrolyte is a solution comprising a salt dissolved in a solvent. In some embodiments, the concentration of the salt in the electrolyte is, or is about, 0.1 M, 0.2 M, 0.3 M, 0.4 M, 0.5 M. 0.6 M, 0.7 M, 0.8 M, 0.9 M, 1 M, 1.1 M, 1.2 M, 1.3 M, 1.5 M, 1.6 M, 1.8 M or 2 M, or any range of values therebetween.

The electrolyte further includes a performance improver, wherein the performance improver comprises a halogenated heteroaryl compound. In some embodiments, the electrolyte comprises a weight percent of total performance improvers of, of about, of at most, or of at most about, 35 wt %, 30 wt %, 25 wt %, 20 wt %, 15 wt %, 12 wt %, 10 wt %, 9 wt %, 8 wt %, 7 wt %, 6 wt %, 5 wt %, 4 wt %, 3 wt %, 2 wt %, 1 wt %, 0.5 wt % or 0.1 wt %, or any range of values therebetween. In some embodiments, the electrolyte comprises a weight percent of each performance improver (e.g., a halogenated heteroaryl compound) of, of about, of at most, or of at most about, 25 wt %, 20 wt %, 15 wt %, 12 wt %, 10 wt %, 9 wt %, 8 wt %, 7 w1%, 6 wt %, 5 wt %, 4 wt %, 3 wt %, 2 wt %, 1 wt %, 0.5 wt %, 0.2 wt % or 0.1 wt %, or any range of values therebetween.

### Halogenated Performance Improvers

As described herein, the electrolyte includes a performance improver comprising a halogenated heteroaryl compound. In some embodiments, the halogenated heteroaryl compound may comprise a nitrogen-containing aromatic heterocycle with at least one halogen atom bonded to the aromatic ring. In some embodiments, the nitrogen-containing aromatic heterocycle can be, for example, a pyridine, a pyrimidine, a pyrazine, or a pyridazine.

Without wishing to be limited by theory, it is thought that the surface of the activated carbon electrode of the energy storage device comprises various oxygen-containing functional groups, such as carbonyl groups and carboxyl functional groups. These oxygen-containing surface functional groups may be highly reactive and can undergo reactions with the electrolyte at high voltages and high temperatures. It is believed that halogenated heteroaryl compounds may displace the oxygen containing functional groups at the surface of an electrode. Further, the halogenated heteroaryl compound may be stabilized through a resonance effect that minimizes the probability of subsequent displacement of the halogenated heteroaryl compound from the surface of an electrode (e.g., an activated carbon electrode).

In some embodiments, a halogenated heteroaryl compound can be a compound of Formula (I) or Formula (II) having the structures.

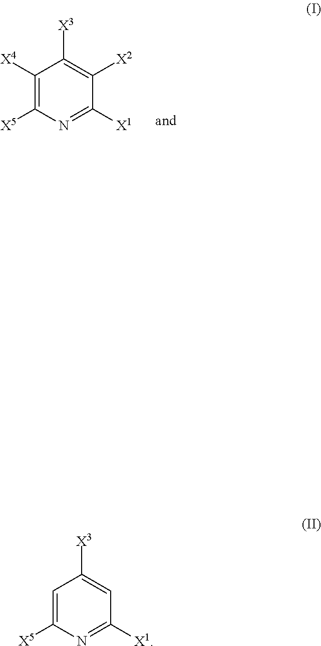

In some embodiments, each X¹, X², X³, X⁴ and X⁵ (e.g., each X¹, X³ and X⁵ for Formula (II)) is independently H or a halogen, provided that at least one of X¹, X², X³, X⁴ and X⁵ is a halogen. In some embodiments, a halogen can be selected from fluoro, chloro, bromo, or iodo. In some embodiments, the compound of Formula (I) is not pyridine. In some embodiments, X¹ is H or a halogen. In some embodiments, X¹ is H, fluoro, chloro, bromo, or iodo. In some embodiments, X¹ is fluoro, chloro, bromo, or iodo. In some embodiments, X¹ is H, chloro, bromo, or iodo. In some embodiments, X¹ is H, fluoro, bromo, or iodo. In some embodiments, X¹ is H, fluoro, chloro, or iodo. In some embodiments, X¹ is H, fluoro, chloro, or bromo. In some embodiments, X² is H or a halogen. In some embodiments, X² is H, fluoro, chloro, bromo, or iodo. In some embodiments, X² is fluoro, chloro, bromo, or iodo. In some embodiments, X² is H, chloro, bromo, or iodo. In some embodiments, X² is H, fluoro, bromo, or iodo. In some embodiments, X² is H, fluoro, chloro, or iodo. In some embodiments, X² is H, fluoro, chloro, or bromo. In some embodiments, X³ is H or a halogen. In some embodiments, X³ is H, fluoro, chloro, bromo, or iodo. In some embodiments, X³ is fluoro, chloro, bromo, or iodo. In some embodiments, X³ is H, chloro, bromo, or iodo. In some embodiments, X³ is H, fluoro, bromo, or iodo. In some embodiments, X³ is H, fluoro, chloro, or iodo. In some embodiments, X³ is H, fluoro, chloro, or bromo. In some embodiments, X⁴ is H or a halogen. In some embodiments, X⁴ is H, fluoro, chloro, bromo, or iodo. In some embodiments, X⁴ is fluoro, chloro, bromo, or iodo. In some embodiments, X⁴ is H, chloro, bromo, or iodo. In some embodiments, X⁴ is H, fluoro, bromo, or iodo. In some embodiments, X⁴ is H, fluoro, chloro, or iodo. In some embodiments, X⁴ is H, fluoro, chloro, or bromo. In some embodiments, X⁵ is H or a halogen. In some embodiments, X⁵ is H, fluoro, chloro, bromo, or iodo. In some embodiments, X⁵ is fluoro, chloro, bromo, or iodo. In some embodiments, X⁵ is H, chloro, bromo, or iodo. In some embodiments, X⁵ is H, fluoro, bromo, or iodo. In some embodiments, X⁵ is H, fluoro, chloro, or iodo. In some embodiments, X⁵ is H, fluoro, chloro, or bromo.

For example, in some embodiments compounds of Formula (I) may include

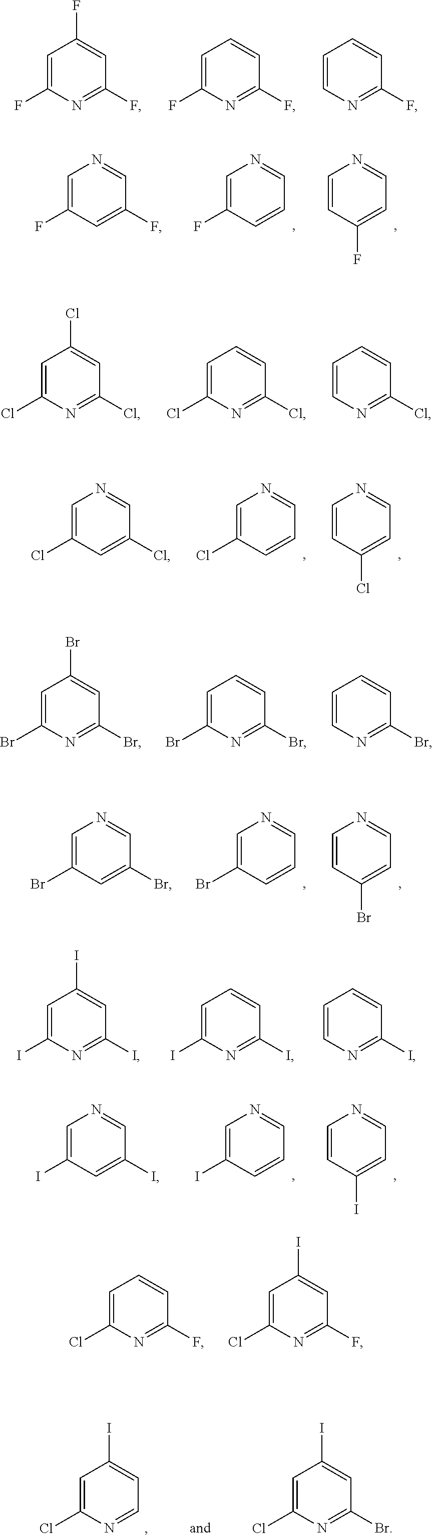

In some embodiments, the halogenated heteroaryl compound is added to the electrolyte of the energy storage device. In some embodiments, more than one species of a halogenated heteroaryl compound can be present in the electrolyte. In some embodiments, the halogenated heteroaryl compound is added to at least one electrode or electrode film of the energy storage device. In some embodiments, more than one species of a halogenated heteroaryl compound can be present in at least one electrode or electrode film. In some embodiments, the electrode or electrode film comprising the halogenated heteroaryl compound is placed into the electrolyte solution.

### EXAMPLES

Example embodiments of the present disclosure, including processes, materials and/or resultant products, are described in the following examples.

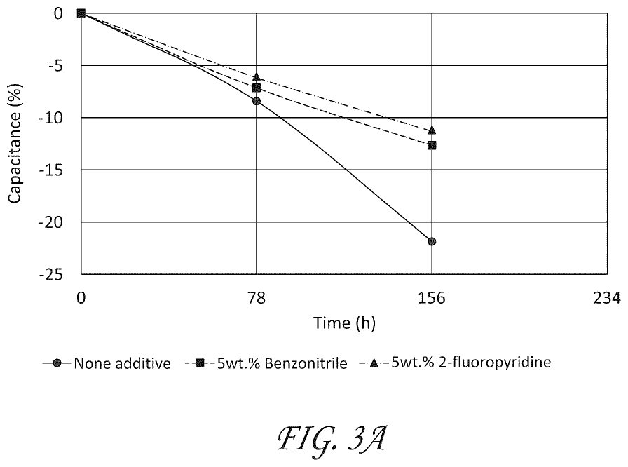

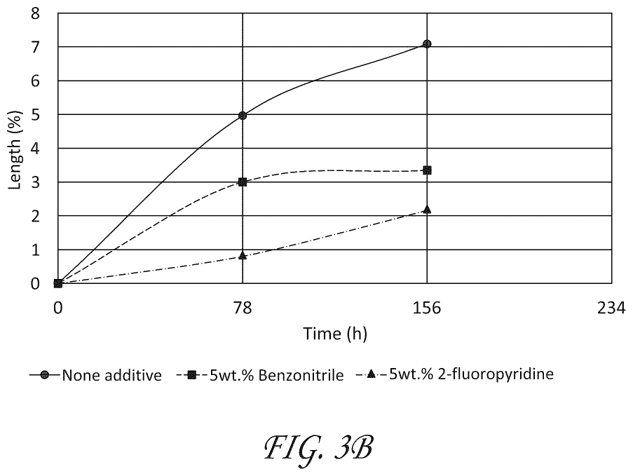

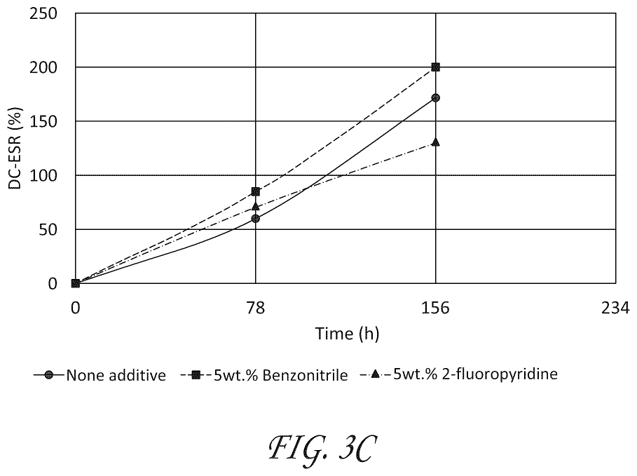

The capacitors described herein and in FIGS. 3A-6D include electrodes formed from an aluminum foil with an electrode film comprising an active material of activated carbon; a binder mixture of a styrene-butadiene rubber, poly(tetrafluoroethylene), carboxymethyl cellulose and poly(vinylpyrrolidone) mixture, and a conductive material of carbon black; a solvent of acetonitrile; and a quaternary ammonium salt.

### Example 1

FIGS. 3A-3C show the change in capacitance, cell length and DC-ESR, respectively, of capacitors tested at 3.2 V and 75° C. for 156 hours, wherein capacitors with 1) control electrolyte (i.e., “none additive”), 2) electrolyte with 5 wt. % benzonitrile as a performance improver and 3) 5 wt. % of the halogenated heteroaryl compound 2-fluoropyridine as a performance improver were compared. FIGS. 3A-3C demonstrate capacitors that include 2-fluoropyridine as a performance improver showed decreased gas generation (FIG. 3B), increased retention of capacitance (FIG. 3A), and suppression of DC-ESR (FIG. 3C) when compared to control or benzonitrile performance improver devices.

### Example 2

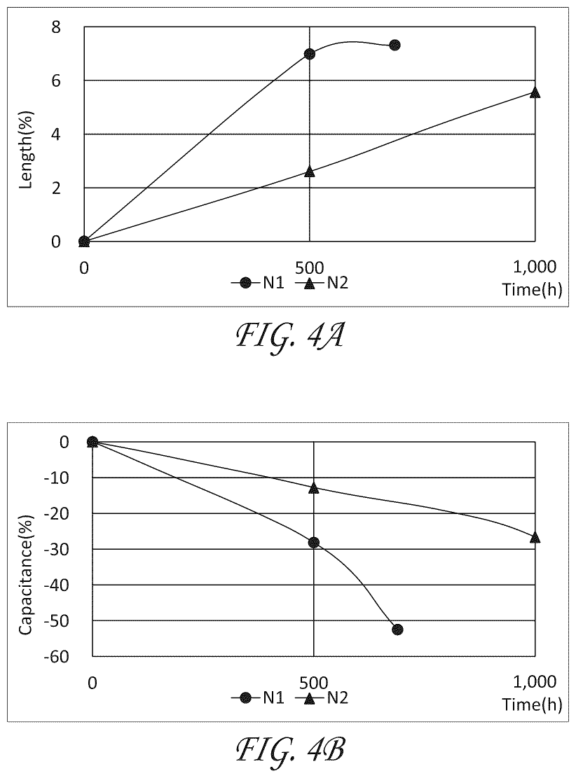

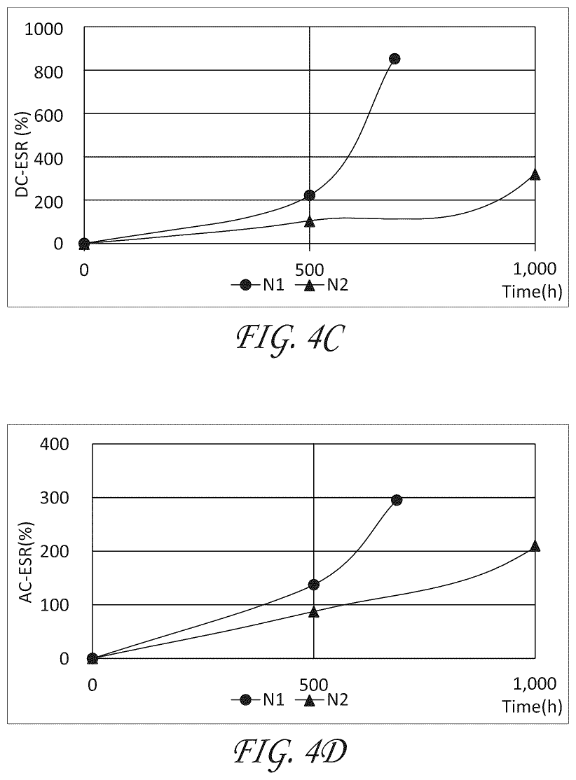

FIGS. 4A-4D show the change in cell length, capacitance, DC-ESR and AC-ESR, respectively, of capacitors tested at 3.2 V and 65° C. for 1000 hours, wherein capacitors with 1) control electrolyte (i.e., “N1”) and 2) an electrolyte with 3 wt. % of the halogenated heteroaryl compound 2-fluoropyridine as a performance improver (“N2”) were compared. FIGS. 4A-4D demonstrate capacitors that include 3 wt. % of a 2-fluoropyridine as a performance improver showed decreased gas generation (FIG. 4A), improved capacitance reduction rates (FIG. 4B), and improved resistance increase rates (FIGS. 4C and 4D) when compared with devices having no performance improver added.

### Example 3

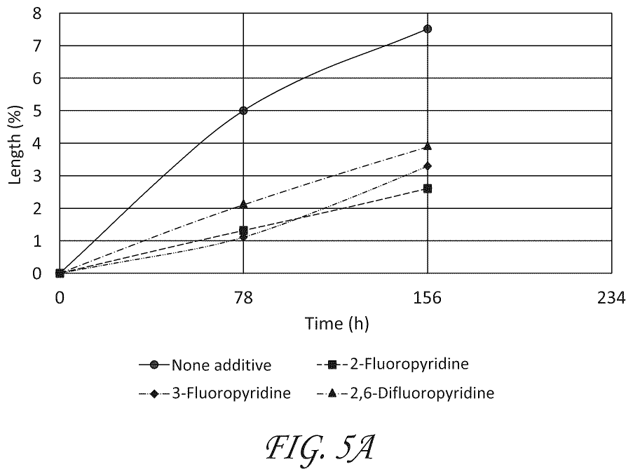

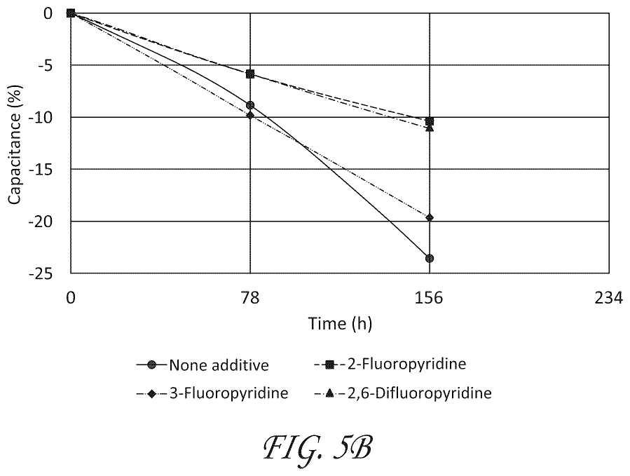

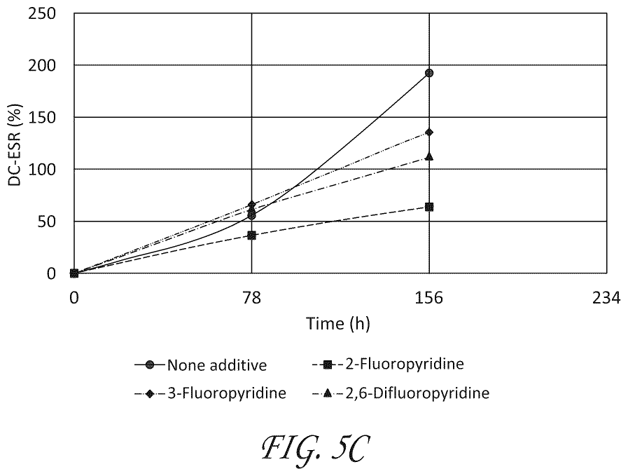

FIGS. SA-5C show the change in cell length, capacitance and DC-ESR, respectively, of capacitors tested at 3.2 V and 75° C. for 156 hours with of different heteroaryl performance improvers, wherein capacitors were tested with 1) control electrolyte (i.e., “none additive”), and electrolytes with 2) halogenated heteroaryl compound 2-fluoropyridine, 3) halogenated heteroaryl compound 3-fluoropyridine or 4) halogenated heteroaryl compound 2,6-difluoropyridine as performance improvers. All capacitors with heteroaryl performance improvers showed decreased gas generation (FIG. 5A), improved capacitance reduction rates (FIG. 5B), and improved resistance increase rates (FIG. SC) when compared with control devices.

### Example 4

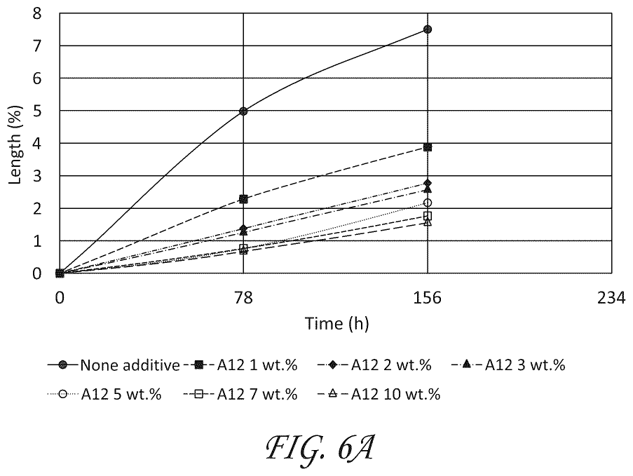

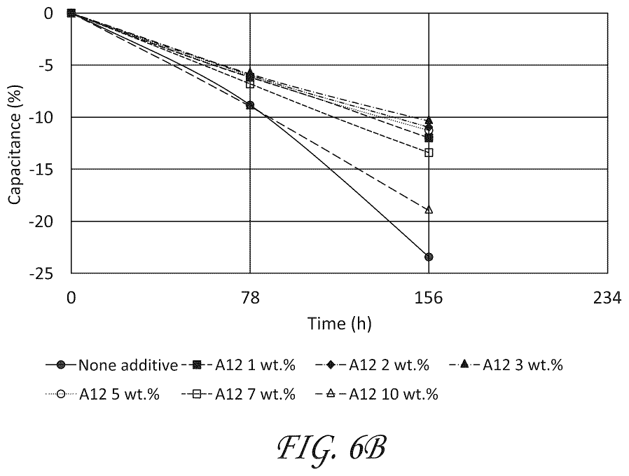

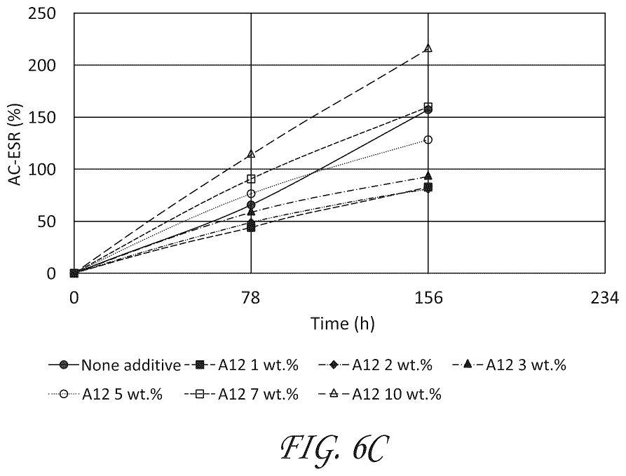

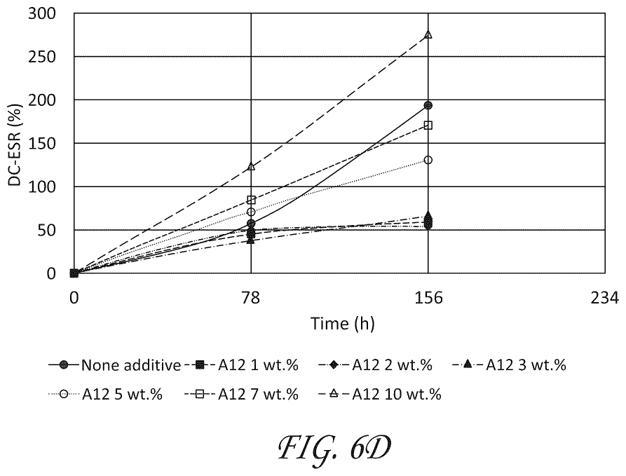

FIGS. 6A-6D show the change in cell length, capacitance, DC-ESR and AC-ESR, respectively, of capacitors tested at 3.2 V and 75° C. for 156 hours with varying concentrations of the halogenated heteroaryl compound 2-fluoropyridine performance improver (i.e., “A12”), wherein capacitors were tested with 1) control electrolytes (i.e., “none additive”), and electrolytes with 2) 1 wt. %, 3) 2 wt. %, 4) 3 wt. %, 5) 5 wt. %, 6) 7 wt. %, and 7) 10 wt. % of 2-fluoropyridine. Capacitors having between about 1 wt. % and about 10 wt. % of a 2-fluoropyridine performance improver showed decreased gas generation (FIG. 6A), and improved capacitance reduction rates (FIG. 6B) when compared with devices having no performance improver added. Capacitors having between about 1 wt. % and about 7 wt. % of a 2-fluoropyridine performance improver showed improved resistance increase rates (FIGS. 6C and 6D) when compared with devices having no performance improver added.

While certain embodiments have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result.

The scope of the present disclosure is not intended to be limited by the specific disclosures of embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

While certain embodiments have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

1. An electric double layer capacitor, comprising:
a cathode;
an anode;
a separator disposed between the cathode and the anode;
an electrolyte, comprising:
a salt;
a solvent; and
a performance improver, wherein the performance improver comprises a halogenated heteroaryl compound; and
a housing, wherein the cathode, anode, separator and electrolyte are disposed within the housing.

2. The electric double layer capacitor of claim 1, wherein the halogenated heteroaryl compound comprises a compound of Formula (I) having the structure: 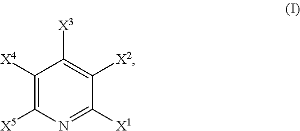
wherein each X¹, X², X³, X⁴ and X⁵ is independently H or a halogen, provided that at least one of X¹, X², X³, X⁴ and X⁵ is a halogen.

3. The electric double layer capacitor of claim 2, wherein the halogen is selected from the group consisting of —F, —Cl, —Br, and —I.

4. The electric double layer capacitor of claim 2, wherein the compound of Formula (I) is a compound selected from the group consisting of:

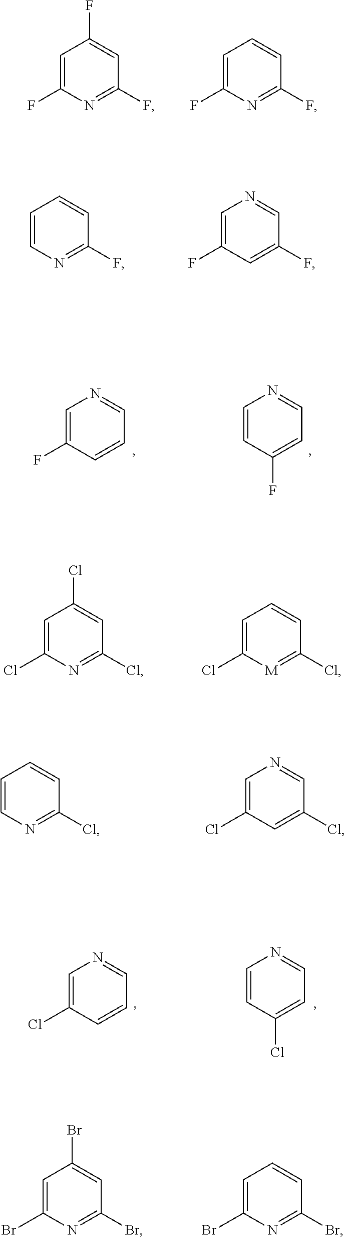

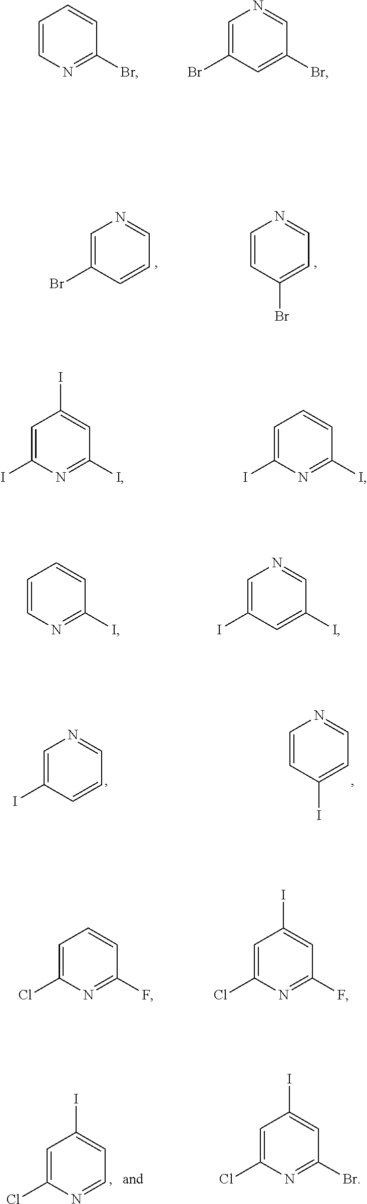

5. The electric double layer capacitor of claim 2, wherein the compound of Formula (I) is selected from the group consisting of:

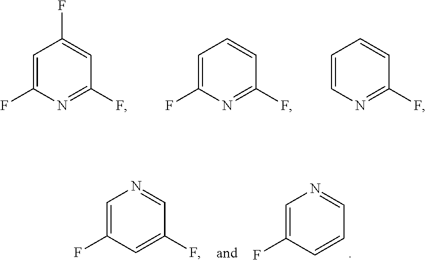 [SMILES: Fc1cc(F)nc(F)c1 ; Fc1cccc(F)n1 ; Fc1ccccn1 ; Fc1cncc(F)c1 ; Fc1cccnc1]

6. The electric double layer capacitor of claim 2, wherein the compound of Formula (I) is

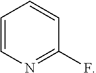

7. The electric double layer capacitor of claim 2, wherein the compound of Formula (I) is

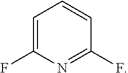

8. The electric double layer capacitor of claim 2, wherein the compound of Formula (I) is

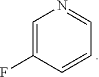

9. The electric double layer capacitor of claim 1, wherein the electrolyte comprises about 0.1 wt % to about 30 wt % of the performance improver.

10. The electric double layer capacitor of claim 1, wherein the electrolyte comprises about 0.5 wt % to about 15 wt % of the compound of Formula (I).

11. The electric double layer capacitor of claim 1, wherein the salt comprises a compound selected from the group consisting of a tetraethyl ammonium salt, a triethylmethyl ammonium salt, a tetrabutylammonium salt, a tetraethylphosphonium salt, a tetrapropylphosphonium salt, a tetrabutylphosphonium salt, a tetrahexylphosphonium salt, a lithium salt, a trimethylethyl ammonium (TMEA) salt, a diethyldimethyl ammonium (DEDMA) salt, a diethyl-methylmethoxyethyl ammonium (DEME) salt, a Tetramethyl ammonium (TMA) salt, a piperidine-1-spiro-1′-pyrrolidinium (SPP) salt, a spiro-(1,1′)-bipyrrolidinium (SBP) salt, a 1,1-Dimethylpyrrolidinium (DMP) salt, a Dimethylammonium (DMA) salt, a 1-Ethyl-1-methylpyrrolidinium (EMP) salt, a 1-Methyl-1-propylpyrrolidinium (MPP) salt, a 1-Butyl-1-methylpyrrolidinium (BMP) salt, a 1-Ethyl-3-methylimidazolium (EMI) salt, a 1-Methyl-3-propylimidazolium (PMI) salt, a 1-Butyl-3-methylimidazolium (BMI) salt, and combinations thereof.

12. The electric double layer capacitor of claim 1, wherein the salt is a quaternary ammonium salt.

13. The electric double layer capacitor of claim 1, wherein the solvent comprises a compound selected from the group consisting of acetonitrile, gamma-butyrolactone, dimethoxyethane, N,N,-dimethylformamide, hexamethyl-phosphorotriamide, tetrahydrofuran, 2-methyltetra-hydrofuran, dimethyl sulfoxide, dimethyl sulfite, sulfolane, nitromethane, dioxolane, ethylene carbonate (EC), propylene carbonate (PC), vinyl ethylene carbonate (VEC), vinylene carbonate (VC), fluoroethylene carbonate (FEC), dimethyl carbonate (DMC), diethyl carbonate (DEC), ethyl methyl carbonate (EMC), capronitrile, valeronitrile, butyronitrile, propionitrile, methyl ethyl sulfone, methyl isopropyl sulfone, ethyl isobutyl sulfone, isopropyl-s-butyl sulfone, butyl isobutyl sulfone, and combinations thereof.

14. The electric double layer capacitor of claim 1, wherein the solvent comprises acetonitrile.

15. The electric double layer capacitor of claim 1, wherein the energy storage device is configured to maintain at least about 80% of its initial capacitance when operating at about 3.2 V for a period of about 1000 hours at a temperature of about 65° C.

16. The electric double layer capacitor of claim 1, wherein the energy storage device is configured to grow at most about 4% of its initial length when operating at about 3.2 V for a period of about 1000 hours at a temperature of about 65° C.

17. The electric double layer capacitor of claim 1, wherein the energy storage device is configured to maintain at most about 175% of its initial DC-ESR when operating at about 3.2 V for a period of about 1000 hours at a temperature of about 65° C.

18. The electric double layer capacitor of claim 1, wherein the energy storage device is configured to maintain at most about 175% of its initial AC-ESR when operating at about 3.2 V for a period of about 1000 hours at a temperature of about 65° C.

19. A method of fabricating an electric double layer capacitor, comprising:
disposing a cathode, an anode, a separator, an electrolyte and a performance improver within a housing, wherein the performance improver comprises a halogenated heteroaryl compound.

20. The method of claim 19, wherein prior to the disposing step at least one of the cathode and anode comprises the performance improver.

21. The method of claim 19, wherein prior to the disposing step the electrolyte comprises the performance improver.

22. An electric double layer capacitor electrode film, comprising:
an active material;
a binder; and
a performance improver, wherein the performance improver comprises a halogenated heteroaryl compound.
